SEQUENCE LISTING (2 sequences)

SEQ ID NO: 1 - protein, 15 aa, Artificial Sequence
  Description of Artificial Sequence Synthetic peptide
CINRKIPIQRLESYT

SEQ ID NO: 2 - protein, 99 aa, Homo sapiens
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

(19) United States
(12) Patent Application Publication
(10) Pub. No.: US 2016/0272702 A1
(43) Pub. Date: Sep. 22, 2016

(54) ANTI-CCL8 THERAPY FOR BREAST CANCER

(71) Applicant: University of South Carolina, Columbia, SC (US)

(72) Inventors: Hippokratis Kiaris, Irmo, SC (US); Eleni Farmaki, Columbia, SC (US); Ioulia Chatzistamou, Irmo, SC (US); Vimala Kaza, Lexington, SC (US)

(21) Appl. No.: 15/072,643

(22) Filed: Mar. 17, 2016

Related U.S. Application Data
(60) Provisional application No. 62/134,820, filed on Mar. 18, 2015.

(51) Int. Cl. C07K 16/24; C12Q 1/68; C07K 16/30; A61K 45/06; A61K 39/395

(52) U.S. Cl. CPC C07K 16/24; A61K 45/06; A61K 39/39558; C07K 16/3015; C12Q 1/686; C07K 2317/76; A61K 2039/505

(57) ABSTRACT

Methods and materials for prevention of migration of breast cancer cells are described. Methods include inhibition of CCL8 activity in the area of the breast cancer cells. One method includes delivery of an anti-CCL8 antibody or an anti-CCL8 antibody expressing vector to an area including the breast cancer cells, e.g., delivery to a subject in need thereof in an effective amount. Another method includes inhibition of expression of CCL8 in a subject, for instance via silencing of the CCL8 gene.

### CROSS REFERENCE TO RELATED APPLICATION

This application claims filing benefit of U.S. Provisional Patent Application Ser. No. 62/134,820 entitled “Anti-Ccl8 Therapy for Metastatic Breast Cancer” having a filing date of Mar. 18, 2015, which is incorporated herein by reference for all purposes.

### GOVERNMENT SUPPORT CLAUSE

This invention was made with government support under 5P30GM103336-O2 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 25, 2016, is named USC-470(1150)_SL.txt and is 2 KB in size.

### BACKGROUND

Tumor cell dissemination reflects the collective outcome of multiple events that include the invasion of the cancer cells into the surrounding stroma, subsequently their intravasation and entrance into circulation, and ultimately their extravasation and seeding into the sites of secondary growth. It is conceivable that interference with the pathways that promote the dissemination of the cancer cells will inhibit metastases providing tools for disease management. Directed cell migration towards specific gradients of chemoattractive factors may provide a model to explain the spread of cancer cells and the initiation of the metastatic process. While this mechanism appears attractive in explaining cancer cell dissemination, details remain elusive, and useful treatment protocols based upon the mechanism likewise remain elusive.

Chemokines are a superfamily of small, cytokine-like proteins that are resistant to hydrolysis, promote neovascularization or endothelial cell growth inhibition, induce cytoskeletal rearrangement, activate or inactivate lymphocytes, and mediate chemotaxis through interactions with G-protein coupled receptors. Chemokines can mediate the growth and migration of host cells that express their receptors.

CCL8 is a small cytokine belonging to the CC chemokine family that attracts monocytes, lymphocytes, basophils and eosinophils. The processed form (generally referred to as MCP-2) is understood to activate many different immune cells, including mast cells, eosinophils and basophils implicated in allergic responses, and monocytes, T cells, and NK cells that are involved in the inflammatory response, and inhibits the chemotactic effect most predominantly of CCL7, but also of CCL2 and CCL5. CCL8 elicits its effects by binding to several different chemokine cell surface receptors including CCR1, CCR2B and CCR5. CCL8 can bind heparin and is believed to play a role in neoplasia and inflammatory host responses.

What are needed in the art are methods and materials for prevention of tumor cell dissemination. Methods and materials for use in aggressive cancers, such as triple-negative breast cancers would be of great benefit.

### SUMMARY

According to one embodiment, disclosed is a method for preventing the migration of breast cancer cells. More specifically, the method includes inhibiting the activity of CCL8 is the area of the breast cancer cells. For instance, the breast cancer cells can include estrogen-independent cells and, in one embodiment, triple negative breast cancer cells.

Also disclosed is a method for treatment of breast cancer that includes inhibiting the activity of CCL8 in a subject suffering from breast cancer. In one embodiment, the method can include determining the level of CCL8 in the subject prior to the treatment process, which can provide information with regard to epithelial-mesenchymal transition and initiation of metastasis in the subject.

Also disclosed is a pharmaceutical composition that can include an anti-CCL8 antibody in conjunction with a pharmaceutical carrier that can be used in a breast cancer treatment method as disclosed herein.

### BRIEF DESCRIPTION OF THE DRAWINGS

The present invention may be better understood with reference to the accompanying figures, in which:

FIG. 1 presents a diagrammatic illustration of a breast cancer dissemination process in which a gradient of CCL8 is self-sustained to promote dissemination of the breast cancer cells.

FIG. 2A schematically presents the genomic organization of the cytokine clusters that harbor CCL8 in mouse and CCL8 in humans.

FIG. 2B illustrates CCL8 expression in mouse and human fibroblasts following exposure to breast cancer cells' conditioned media.

FIG. 2C presents experimental results showing that CCL8 is the only cytokine in both the human 17q12 cluster and the mouse 11C cluster that specifically responds to breast cancer cell media in both mouse and human fibroblasts.

FIG. 3A graphically illustrates the chemoattraction of various breast cancer cells for fibroblasts upon increase in the amounts of CCL8 in transwells in vitro.

FIG. 3B illustrates the vimentin (Vm) levels in EO771 cells after treatment with CCL8 suggesting the induction of epithelial-mesenchymal transition (EMT).

FIG. 4A illustrates a drop in EO771 tumor onset in wild type (wt) mice following administration of a neutralizing antibody for CCL8.

FIG. 4B illustrates the effect on migrated cells upon inhibition of CCL8 by a neutralizing antibody for CCL8 in RAW macrophages.

FIG. 4C illustrates delay of EO771 tumor onset in mice following genetic ablation of CCL8.

FIG. 4D presents CCL8 levels in EO771 tumors developed in wt and CCL8KO mice and in EO771 cells cultured in vitro.

FIG. 4E illustrates correlation between serum CCL8 levels and tumor volume in EO771 breast cancer-bearing mice.

FIG. 4F compares EO771 tumors in CCL8KO and wt mice.

FIG. 4G illustrates SMA immunostaining and Van Gieson staining of EO771 tumors in control and CCL8 knockout (CCL8KO) mice.

FIG. 5 illustrates an association between high CCL8 expression and negative prognosis in breast cancer patients.

FIG. 6 graphically illustrated the migration rates of MDA-MB-231 breast cancer cells in the presence of various antibodies.

FIG. 7 illustrates migration of MDA-MB-231 breast cancer cells in the presence of various cytokines encoded by the 17q12 gene both alone and in the presence of an anti-CCL8 antibody.

FIG. 8 presents images illustrating that the presence of anti-CCL8 antibody inhibits migration and invasion of breast cancer cells toward adjacent matrigel nodules containing CCL8-producing fibroblasts.

FIG. 9 provides the sequence of human CCL8 and includes details of several elements of the sequence including the antigenic peptide described further herein.

### DETAILED DESCRIPTION

The following description and other modifications and variations to the presently disclosed subject matter may be practiced by those of ordinary skill in the art, without departing from the spirit and scope of the present invention. In addition, it should be understood that aspects of the various embodiments may be interchanged both in whole and in part. Furthermore, those of ordinary skill in the art will appreciate that the following description is by way of example only, and is not intended to limit the disclosed subject matter.

In general, disclosed herein are methods and materials for prevention of migration of breast cancer cells. Disclosed methods and materials have been developed through recognition that a gradient of increasing CCL8 concentration is maintained from the epithelium towards the stroma and this gradient can be encouraged by breast cancer cells and instrumental for cancer cell dissemination. In one particular embodiment, the disclosed methods can be utilized in treatment of breast cancer.

The terms “treat,” “treating” or “treatment” as used herein, refers to a method of alleviating or abrogating a disorder and/or its attendant symptoms. The terms “prevent”, “preventing” or “prevention,” as used herein, refer to a method of barring a subject from acquiring a disorder and/or its attendant symptoms. In certain embodiments, the terms “prevent,” “preventing” or “prevention” refer to a method of reducing the risk of acquiring a disorder and/or its attendant symptom, e.g., metastatic breast cancer.

While not wishing to be bound to any particular theory, it is believed that CCL8 production by stromal fibroblasts can be upregulated in response to signals elicited by breast cancer cells, especially those from triple-negative breast cancers. Moreover, CCL8 can operate as a potent chemoattractant for various stromal and breast cancer cells, suggesting a role for CCL8 in the promotion of metastases. The presently disclosed methods and materials are directed to inhibition of CCL8 activity, which can inhibit the onset of cell dissemination from a tumor and can cause the development of well-confined lesions of increased cellularity and diminished stroma. The presently disclosed findings are consistent with the concept of an establishment of a self-sustained CCL8 gradient between cancer cells and fibroblasts that ultimately can promote breast cancer metastasis if left unchecked. Disruption of this gradient as disclosed herein can provide means for the management of breast cancers, and in one particular embodiment estrogen-independent breast cancers such as triple-negative breast cancers.

It is hypothesized that soluble factors produced by the stroma in response to signals elicited by the epithelium may facilitate the establishment and maintenance of gradients that can stimulate the directional migration of cancer cells promoting tumor cell dissemination. Considering that the stromal cells produce these chemoattractive factors makes these gradients self-sustained, since the soluble factors' concentration will be higher distally than proximally to the cancer cells.

As described further herein, the chemokine CCL8 has emerged as a good candidate for participating in the establishment of such gradients because cancer cells have been found to stimulate CCL8 production in adjacent stromal fibroblasts. Furthermore CCL8 has been identified by proteomic analyses as a stroma-derived protein in human cancers and its levels have been determined to be significantly elevated in the plasma of breast cancer-prone mice during disease progression. Finally, preliminary results have identified CCL8 as a target of Notch signaling, a pathway with strong paracrine activity.

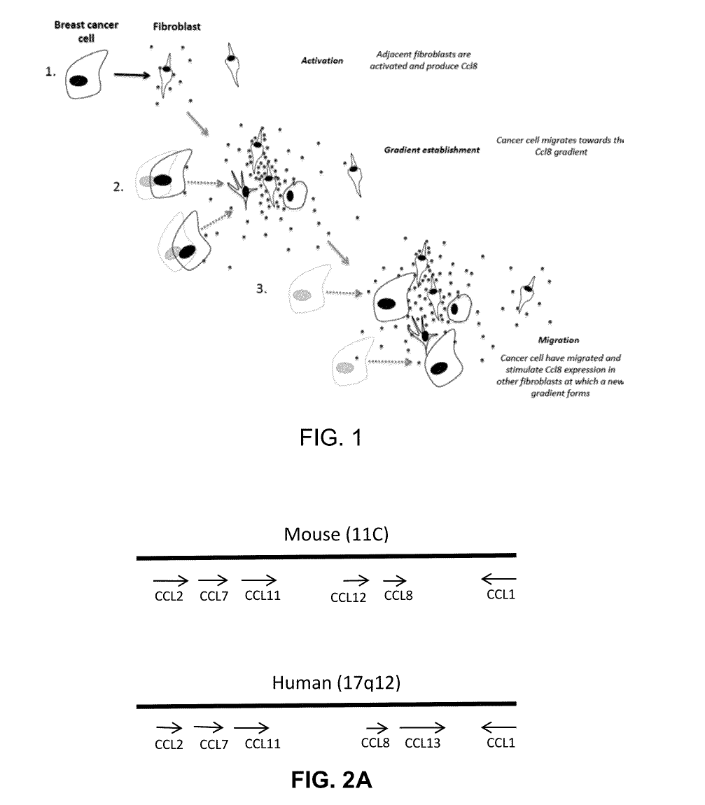

A model for the role of CCL8 in cancer cell dissemination is illustrated in FIG. 1. As shown, it appears that soluble factors that are produced by the breast cancer cells can act on adjacent stromal fibroblasts to activate CCL8. Subsequently, CCL8 attracts the breast cancer cells that can now activate CCL8 in other fibroblasts generating a self-sustained gradient that ultimately promotes the dissemination of the cancer cells and metastasis. Accordingly, disclosed methods are generally directed to blockade of CCL8 activity so as to prevent cancer cell dissemination. Blockade of Ccl8 activity can provide a strategy for breast cancer management and especially for the inhibition of metastases.

According to the present disclosure, breast cancer cell migration can be prevented by decreasing the activity of CCL8 in an area encompassing the breast cancer cells. CCL8 activity can be decreased either directly, e.g., by delivery of an active agent that can interfere with previously formed CCL8 or indirectly, e.g., by delivery of an active agent that can decrease expression or proper formation of CCL8. In one embodiment, a method can include administering to a subject diagnosed with breast cancer a therapeutically effective amount of the active agent. For instance, the subject can be diagnosed with breast cancer and can exhibit elevated CCL8 expression in the stroma surrounding or in an area near a tumor.

In another embodiment, a method can include determining the level of CCL8 in a tissue from a subject and, if an increased level of CCL8 is detected administering to the subject an agent capable of directly or indirectly decreasing activity of CCL8 in the area. According to one embodiment, determination of the level of CCL8 in a subject can be utilized as a biomarker for epithelial mesenchymal transition.

In one embodiment, CCL8 activity can be blocked by delivery or expression of an anti-CCL8 antibody. As used herein, the term “antibody” refers to immunoglobulin molecules and immunologically active portions of immunoglobulin (Ig) molecules, i.e., molecules that contain an antigen binding site that specifically binds (immunoreacts with) an antigen. The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. By “specifically bind” or “immunoreacts with” is meant that the antibody reacts with one or more antigenic determinants of the desired antigen and does not react (i.e., bind) with other polypeptides or binds at much lower affinity with other polypeptides. The term “antibody” also includes antibody fragments that comprise a portion of a full length antibody, generally the antigen binding or variable region thereof. Examples of antibody fragments include Fab, Fab′, F(ab′)2, and Fv fragments; diabodies; linear antibodies; single-chain antibody (scFv) molecules; and multispecific antibodies formed from antibody fragments. In certain embodiments of the invention, it may be desirable to use an antibody fragment, rather than an intact antibody, to increase tumor penetration, for example. In this case, it may be desirable to use an antibody fragment that has been modified by any means known in the art in order to increase its serum half-life.

The term “monoclonal antibody” as used herein refers to an antibody obtained from a population of substantially homogeneous antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

An anti-CCL8 antibody is one that binds to human CCL8 and preferably blocks (partially or completely) the ability of breast cancer cells to bind or otherwise recognize CCL8. In one embodiment, the anti-CCL8 antibody is a monoclonal antibody. In another embodiment, the anti-CCL8 antibody is a humanized antibody. In another embodiment, the anti-CCL8 antibody is a humanized antibody fragment. “Humanized” forms of non-human antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and/or capacity. Methods for making humanized and other chimeric antibodies are known in the art.

Methods and materials useful for decreasing the activity of CCL8 in an area are not limited to utilization of anti-CCL8 antibodies. CCL8 activity can be decreased by use of other active agents and methods including, without limitation, delivery of a vector capable of expressing or inducing expression of an anti-CCL8 antibody in the area of the breast cancer cells or by preventing expression or proper formation of CCL8 via, e.g., gene silencing.

An expression vector can be any vector that is capable of delivery of nucleotides encoding an anti-CCL8 antibody in a target cell. By way of example, expression vectors can include viral vectors and non-viral vectors. Viral vectors include, but are not limited to, retrovirus vectors, adenovirus vectors, adeno-associated virus vectors, and other large capacity viral vectors, such as herpes virus and vaccinia virus. Also included are any viral families which share the properties of these viruses which make them suitable for use as expression vectors.

A retrovirus is an animal virus belonging to the virus family of Retroviridae, including any types, subfamilies, genus, or tropisms. Examples of methods for using retroviral vectors for gene therapy are described in U.S. Pat. Nos. 4,868,116 and 4,980,286; PCT applications WO 90/02806 and WO 89/07136; the teachings of which are incorporated herein by reference.

Recombinant adenoviruses can achieve high efficiency gene transfer with direct, in vivo delivery to airway epithelium, hepatocytes, vascular endothelium, CNS parenchyma and a number of other tissue sites. Recombinant adenoviruses achieve gene transduction by binding to specific cell surface receptors, after which the virus is internalized by receptor-mediated endocytosis, in the same manner as wild type or replication-defective adenovirus.

Another type of viral vector is based on an adeno-associated virus (AAV). This defective parvovirus can infect many cell types and is nonpathogenic to humans. AAV type vectors can transport about 4 to 5 kb and wild type AAV is known to stably insert into chromosome 19. Vectors which contain this site specific integration property are preferred. One example of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

Molecular genetic experiments with large human herpes viruses have provided a means whereby large heterologous DNA fragments can be cloned, propagated and established in cells permissive for infection with herpes viruses. These large DNA viruses e.g., herpes simplex virus (HSV) and Epstein-Barr virus (EBV), have the potential to deliver fragments of human heterologous DNA>150 kb to specific cells.

Examples of non-Viral vectors include plasmid expression vectors. Plasmid vectors typically include a circular double-stranded DNA loop into which additional DNA segments can be inserted.

In both viral and non-viral expression vectors, the polynucleotide encoding the antibody or antibodies is typically arranged in proximity and orientation to an appropriate transcription control sequence (promoter, and optionally, one or more enhancers) to direct mRNA synthesis. That is, the polynucleotide sequence of interest is operably linked to an appropriate transcription control sequence. Examples of such promoters include: viral promoters such as the immediate early promoter of CMV, LTR or SV40 promoter, polyhedron promoter of baculovirus, E. coli lac or trp promoter, phage T7 and λP_{L} promoter, and other promoters known to control expression of genes in eukaryotic cells or their viruses. The promoters may be a tissue specific promoter.

The expression vector typically also contains a ribosome binding site for translation initiation, and a transcription terminator. The vector optionally includes appropriate sequences for amplifying expression. In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli.

An expression vector can also include additional expression elements, for example, to improve the efficiency of translation. These signals can include, e.g., an ATG initiation codon and adjacent sequences. If desired, the efficiency of expression can be further increased by the inclusion of enhancers appropriate to the cell system in use.

In one embodiment, the expression vector contains an inducible or regulatable expression system. Examples of regulatable expression systems include the ecdysone system, the progesterone system, and the rapamycin system.

According to one embodiment, an agent that inhibits the expression or proper formation of CCL8 can be utilized. For example, an effective amount of an agent or an expression vector that expresses an agent that inhibits the expression of CCL8 can be delivered to a subject in need thereof. For example, the agent can be a functional nucleic acid. Functional nucleic acids are nucleic acid molecules that have a specific function, such as binding a target molecule or catalyzing a specific reaction. The functional nucleic acid molecules can act as inhibitors of a specific activity possessed by a target molecule. Functional nucleic acid molecules can interact with any macromolecule, such as DNA, RNA and polypeptides. Thus, functional nucleic acids can interact with mRNA or the genomic DNA of CCL8 to inhibit activity. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. Examples of functional nucleic acid molecules include siRNA, antisense molecules, aptamers, ribozymes, triplex forming molecules, and external guide sequences.

siRNA is involved in RNA interference (RNAi) which involves a two-step mechanism: an initiation step and an effector step. In the first step, input double-stranded (ds) RNA (siRNA) is processed into small fragments, such as 21-23-nucleotide ‘guide sequences’. RNA amplification occurs in whole animals. Typically, the guide RNAs can be incorporated into a protein RNA complex which is capable of degrading RNA, the nuclease complex, which has been called the RNA-induced silencing complex (RISC). This RISC complex acts in the second effector step to destroy mRNAs that are recognized by the guide RNAs through base-pairing interactions. RNAi involves the introduction by any means of double stranded RNA into the cell which triggers events that cause the degradation of a target RNA. RNAi is a form of post-transcriptional gene silencing.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods include in vitro selection experiments and DNA modification studies using DMS and DEPC. The antisense molecules can generally bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,994,320, 6,046,319, and 6,057,437, all of which are incorporated herein by reference in their entireties.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind a chemokine and block its function. Aptamers can bind very tightly with k_{d} from the target molecule of less than 10⁻¹²M. Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). Representative examples of how to make and use aptamers to bind a variety of different target molecules can be found in the following non-limiting list of U.S. Pat. Nos. 5,476,766, 5,861,254, 6,030,776, and 6,051,698, all of which are incorporated herein by reference in their entireties.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (see, e.g., U.S. Pat. Nos. 5,334,711 and 5,861,288, WO 9858058 and WO 9718312) hairpin ribozymes (see, e.g., U.S. Pat. Nos. 5,631,115 and 6,022,962), and tetrahymena ribozymes (see, e.g., U.S. Pat. Nos. 5,595,873 and 5,652,107). There are also a number of ribozymes that are not found in natural systems, but which have been engineered to catalyze specific reactions de novo (see, e.g., U.S. Pat. Nos. 5,580,967 and 5,910,408). Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,869,253, 5,989,906, and 6,017,756, all of which are incorporated herein by reference in their entireties.

Triplex forming functional nucleic acid molecules are molecules that can interact with either double-stranded or single-stranded nucleic acid. When triplex molecules interact with a target region, a structure called a triplex is formed, in which three strands of DNA form a complex dependant on both Watson-Crick and Hoogsteen base-pairing. Triplex molecules are preferred because they can bind target regions with high affinity and specificity. A triplex forming molecule can generally bind the target molecule with a k_{d} less than 10⁻⁶. Representative examples of how to make and use triplex forming molecules to bind a variety of different target molecules can be found in U.S. Pat. Nos. 5,176,996, 5,683,874, 5,874,566, and 5,962,426, all of which are incorporated herein by reference in their entireties.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162, all of which are incorporated herein by reference in their entireties.

An agent may be administered to a subject according to known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In certain embodiments, an antibody is administered directly to the area of a tumor or cancer tissue, including administration directly to the tumor stroma during invasive procedures. The agent may also be placed on a solid support such as a sponge or gauze for administration and activity against CCL8.

A composition including an active component (e.g., an anti-CCL8 antibody) can be administered in conjunction with an accepted pharmaceutically acceptable carrier. Acceptable carriers include, but are not limited to, saline, buffered saline, glucose in saline. Solid supports, liposomes, nanoparticles, microparticles, nanospheres or microspheres may also be used as carriers for administration of the antibodies.

The appropriate dosage (“therapeutically effective amount”) of the active component (e.g., the anti-CCL8 antibody) can depend, for example, on the particular breast cancer to be treated, the severity and course of the breast cancer, whether the agent is administered for preventive or therapeutic purposes, previous therapy, the patient's clinical history and response to the agent, the type of agent used, and the discretion of the attending physician. An active agent can be administered to a patient at one time or over a series of treatments and may be administered to the patient at any time from diagnosis onwards. An agent may be administered as the sole treatment or in conjunction with other drugs or therapies useful in treating breast cancer.

In one embodiment, a therapeutically effective amount of an agent can be in the range of about 1 ng/kg body weight/day to about 100 mg/kg body weight/day whether by one or more administrations. For example, an anti-CCL8 antibody can be administered in an amount of from about 1 ng/kg body weight/day to about 1 μg/kg body weight/day, or from about 0.5 mg/kg body weight per day to about 50 mg/kg body weight/day, in some embodiments. In other particular embodiments, the amount of an agent administered can be from about, 0.0005 mg/day to about 1000 mg/day or from about 0.1 mg/day to about 500 mg/day in some embodiments. As expected, the dosage will be dependant on the condition, size, age and condition of the patient.

An active agent may be administered, as appropriate or indicated, a single dose as a bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. However, other dosage regimens may be useful. The progress of this therapy is easily monitored by conventional techniques.

In one embodiment, an active agent that decreases the activity of CCL8 in an area can be administered to a subject in need thereof in conjunction with one or more additional therapeutically effective agents. For instance, the active agent can be administered in conjunction with another anti-cancer agent, such as chemotherapy agent. Additional therapeutically effective agents can be administered as a component of the composition that includes the active agent that decreases the activity of CCL8 or in a separate composition, as desired.

As used herein the term “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

A pharmaceutical composition can be formulated to be compatible with its intended route of administration. Examples of routes of administration include parenteral, e.g., intrathecal, intra-arterial, intravenous, intradermal, subcutaneous, oral, transdermal (topical) and transmucosal administration. In certain embodiments, the pharmaceutical composition is administered directly into the tissue surrounding a tumor.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent that delays absorption, for example, aluminum monostearate or gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially, for example, from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the application are dictated by and directly dependent on the unique characteristics of the active compound and the particular therapeutic effect to be achieved, and the limitations inherent in the art of compounding such an active compound for the treatment of individuals.

Disclosed methods and materials can be utilized in one embodiment for therapeutic interference for targeting breast cancer metastasis. Also suggested is a novel paradigm on how dissemination of the cancer cells can be attained beyond the conventional notion of EMT induction and stochastic stimulation of epithelial cell motility. Accordingly, even transient activation of CCL8 levels in peripheral tissues, such as may be caused by inflammation, may attract circulating cancer cells and trigger metastatic seeding. Presently disclosed methods and materials may be beneficial in preventing such metastatic processes.

The present invention may be better understood with reference to the Examples, set forth below.

### EXAMPLE 1

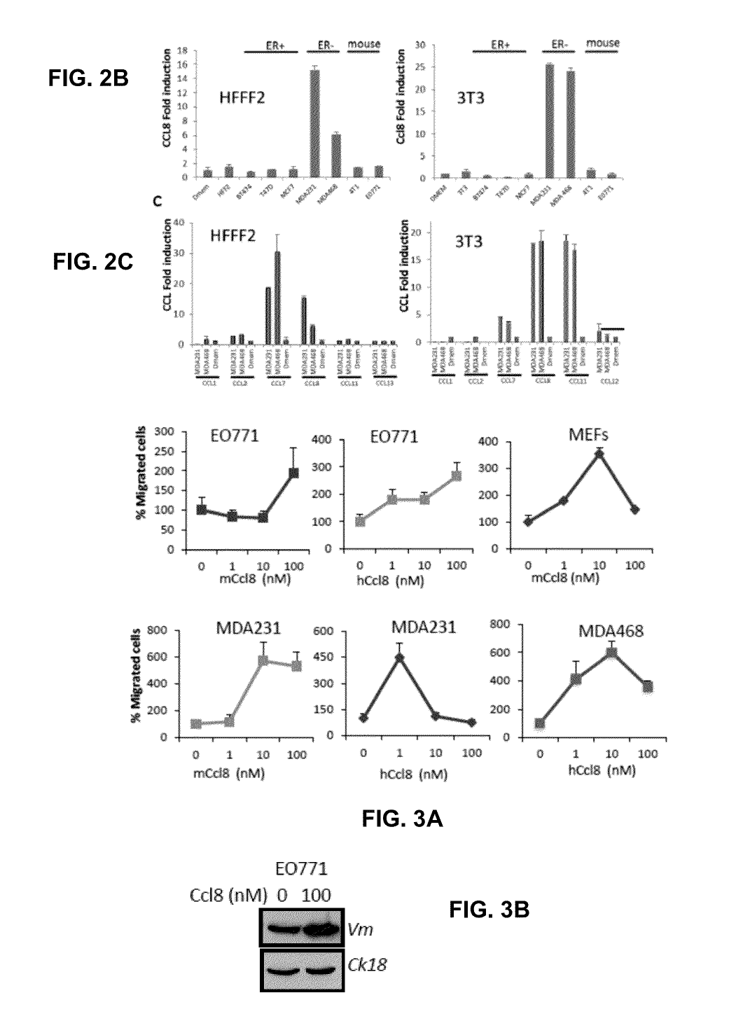

In order to test how breast cancer cells affect CCL8 production in the stroma, human HFFF2 and mouse 3T3 fibroblasts were exposed to conditioned media from a panel of breast cancer cells, both estrogen-dependent and estrogen-independent, and the levels of CCL8 were measured by qPCR. The results (FIG. 2B) showed that media from the triple negative breast cancers (TNBCs) MDA-MB-(MDA) 231 and MDA468 cells were stimulatory for CCL8 in both human and mouse fibroblasts while media from the estrogen-dependent MCF7, BT474, and T47D were not. This suggests that transition to estrogen-independency may be linked to the production of soluble factors that can stimulate CCL8 expression in adjacent fibroblasts.

In view of the fact that the gene encoding for CCL8 belongs to a cluster of chemoattractive cytokines (FIG. 2A) a comparison of how specific the stimulation of CCL8 is as compared to that of the other cytokines that are encoded by genes located in the same genomic cluster was carried out. As shown in FIG. 2C, besides CCL8, CCL7 in human HFFF2 and CCL11 in mouse 3T3 fibroblasts were also induced by the media from TNBCs MDA468 and MDA231 cells. However, as can be seen, CCL8 was the only cytokine that was induced in both mouse and human fibroblasts. This observation argues in favor of the important role of CCL8 in stroma-epithelium interactions, particularly those mediating the communication of TNBC cells with their microenvironment.

The consequences of CCL8 in breast cancer cells and fibroblasts were also examined. Given the strong chemoattractive activity of CCL8 in immune cells such as the macrophages, the assessment of CCL8-induced chemoattraction for various breast cancer cells was determined. As shown in FIG. 3A, both mouse and human CCL8 attracted breast cancer cells of mouse and human origin. Besides further supporting its significant role, the observation that CCL8-induced chemoattraction is retained between the cross species barriers also suggests that human and mouse CCL8 are functionally interchangeable which is of particular value for experimental studies involving cells of different origin. Vimentin overexpression, a marker of epithelial-mesenchymal transition, in EO771 mouse breast cancer cells following CCL8 treatment (FIG. 3B), also supports the instrumental role of CCL8 in triggering a cell migration.

### EXAMPLE 2

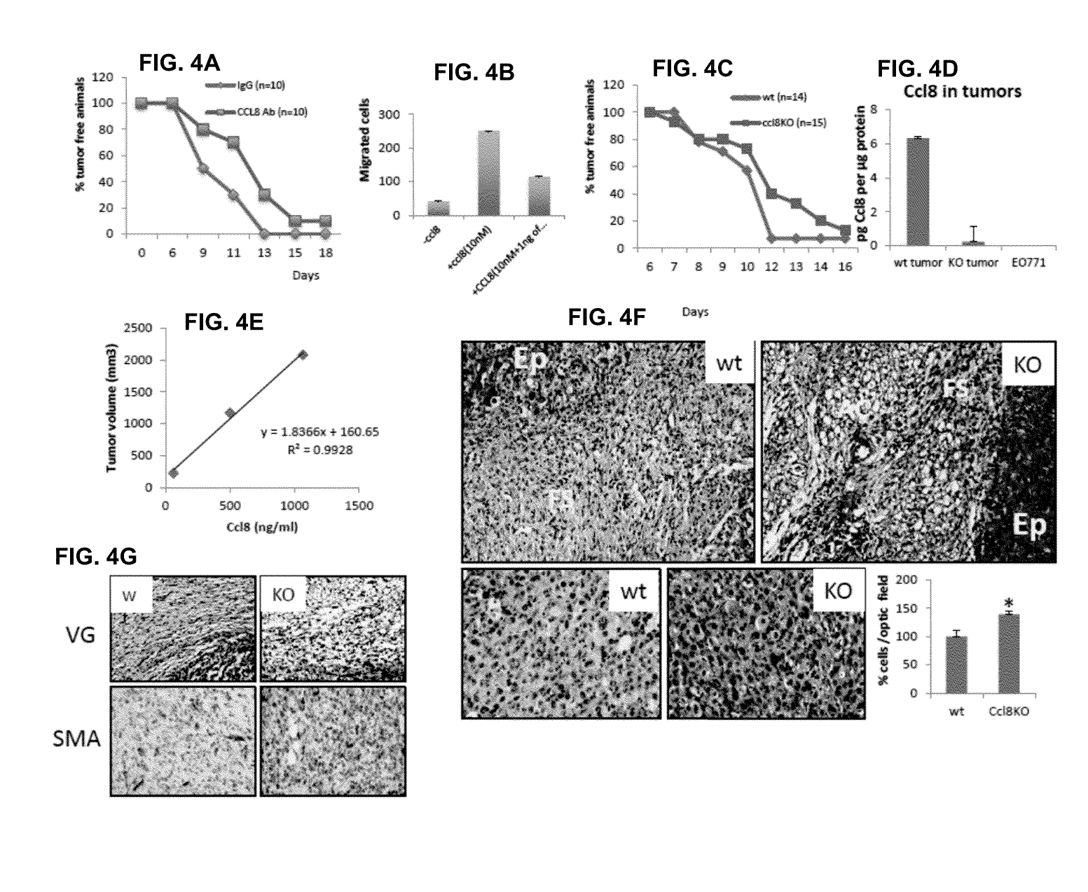

The consequences of CCL8 ablation were determined in vivo in tumor-bearing mice. First CCL8 activity was blocked by a neutralizing antibody administered daily for 1 week in mice following the inoculation of EO771 mouse breast cancer cells in the mammary fat pad of wild type mice. As shown in FIG. 4A, inhibition of CCL8 activity significantly delayed the onset of EO771 breast cancer cells in syngeneic C57BL6 mice.

The effect of genetic ablation of CCL8 in the onset of EO771 tumors in wild type (wt) and CCL8-deficient (CCL8KO) mice was then tested. The CCL8KO mice were generated by the University of California (KOMP repository). Consistently with the effects of antibody-mediated inhibition, genetic deletion of CCL8 also delayed the onset of EO771 tumors (FIG. 4C). Importantly, the source of CCL8 in these cancers was almost exclusively the stroma and not the epithelium as indicated by the virtual absence of CCL8 from the tumors of the Ccl8KO mice (FIG. 4D). Thus, host-derived CCL8 effectively modulated and indeed promoted the tumors' onset. To that end, the levels of CCL8 may reflect the degree of activation of the tumor stroma and can operate as a biomarker for disease progression. This is also supported by the positive correlation between CCL8 levels and tumor volume (FIG. 4E).

Morphological examination of the resulting tumors suggested that stromal CCL8 deficiency not only affected the kinetics but also the morphology of the tumors: EO771 breast cancers that developed in the CCL8KO mice had increased cellularity and better defined borders (FIG. 4F) than the tumors in the wt animals. In addition, tumors in CCL8KO hosts contained poorer stroma (Van Geison staining and smooth muscle actin (SMA) immunostaining, FIG. 4G). Thus, manipulation of CCL8 activity effectively altered tumor's morphology and especially the tumor stroma. Specifically, ablation of CCL8 inhibited the dissemination of the cancer cells suggesting the anti-metastatic activity of Ccl8 suppression.

### EXAMPLE 3

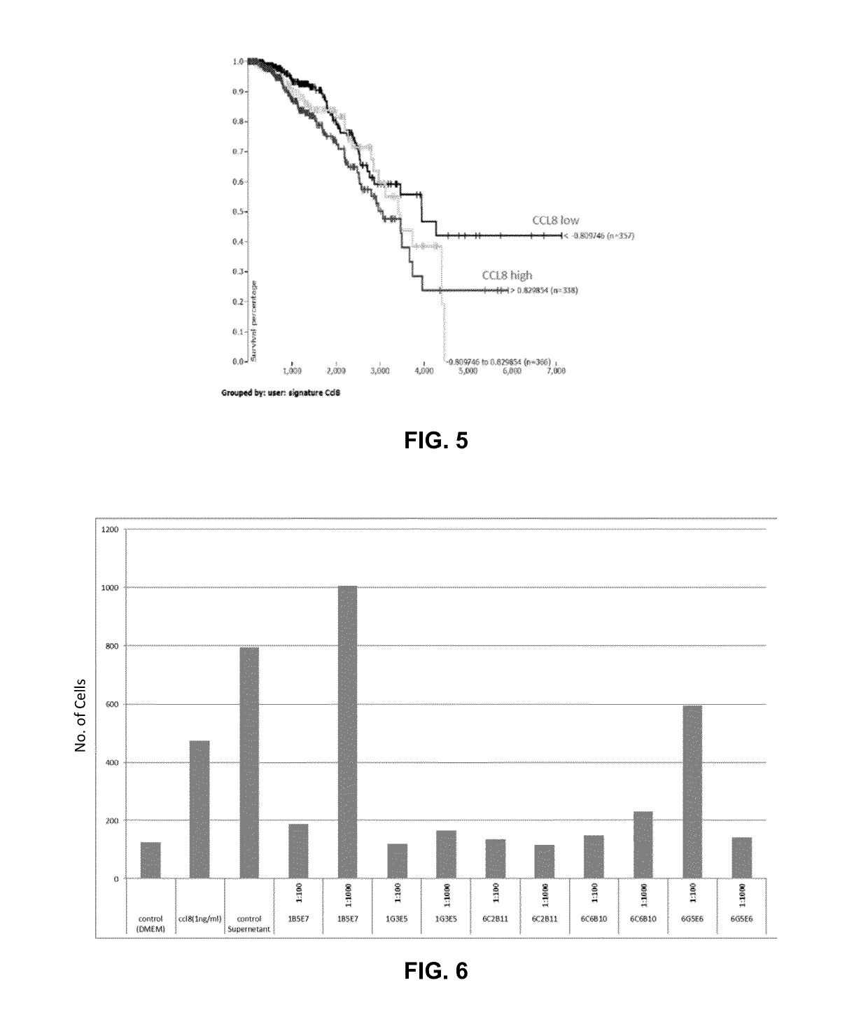

Data mining for CCL8 expression in tumors of breast cancer patients was carried out. The data were obtained by the Cancer Genomics Browser (University of California at Santa Cruz, https://genome-cancer.ucsc.edu/). Analysis of these data suggests that high CCL8 expression is associated with considerably worse prognosis in breast cancer patients (FIG. 5).

### EXAMPLE 4

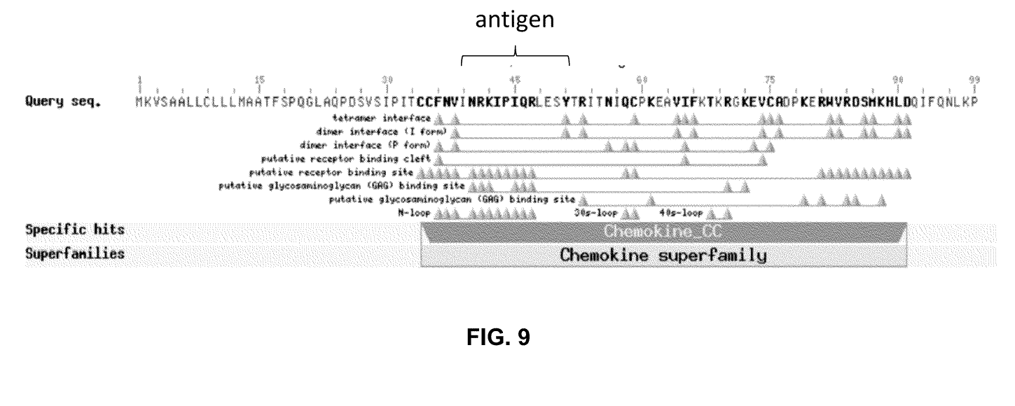

Monoclonal antibodies were prepared against the antigen sequence CINRKIPIQRLESYT (SEQ ID NO.: 1). This antigen has similarity to human CCL7 and CCL11, but not mouse CCL8). This sequence is derived from the human CCL8 sequence (SEQ ID NO.: 2) as illustrated in FIG. 9. The immunogen was a peptide-KLG conjugate developed with a BALB/c mouse host strain. The myeloma type was SP2/0.

Elisa results of the culture supernatant are provided in Table 1, below.

TABLE 1

| | | | | | | | | | | Supernatant |
|---|---|---|---|---|---|---|---|---|---|---|
| Cell | Supernatant Dilution | | | | | | | | | Concentration |
| lines | 1:10 | 1:30 | 1:90 | 1:270 | 1:810 | 1:2430 | Blank | Titer | Isotype | (μg/mL) |
| 1B5E7 | 3.135 | 3.003 | 2.596 | 2.269 | 1.693 | 1.243 | 0.054 | >1:2430 | IgG2b, κ | 16.756 |
| 1G3E5 | 2.856 | 2.769 | 2.644 | 2.394 | 2.009 | 1.351 | 0.054 | >1:2430 | IgG1, κ | 23.582 |
| 6C2B11 | 2.567 | 2.341 | 1.995 | 1.396 | 0.861 | 0.461 | 0.054 | >1:2430 | IgG2a, κ | 4.036 |
| 6C6B10 | 2.531 | 2.429 | 2.283 | 1.968 | 1.455 | 0.885 | 0.054 | >1:2430 | IgG1, κ | 12.419 |
| 6G5E6 | 2.573 | 2.379 | 2.079 | 1.600 | 0.961 | 0.563 | 0.054 | >1:2430 | IgG1, κ | 4.473 |

FIG. 6 graphically illustrates the migration of MDA-MB 231 breast cancer cells with human CCL8 in the transwell chambers for different monoclonal antibodies at a 1:100 and 1:1000 dilution. DMEM, CCL8 (1ng/mL) and supernatant are included as control. As shown, the supernatant from clone 1 G3E5 and 6C6B10 had the highest dose-dependent neutralizing effect.

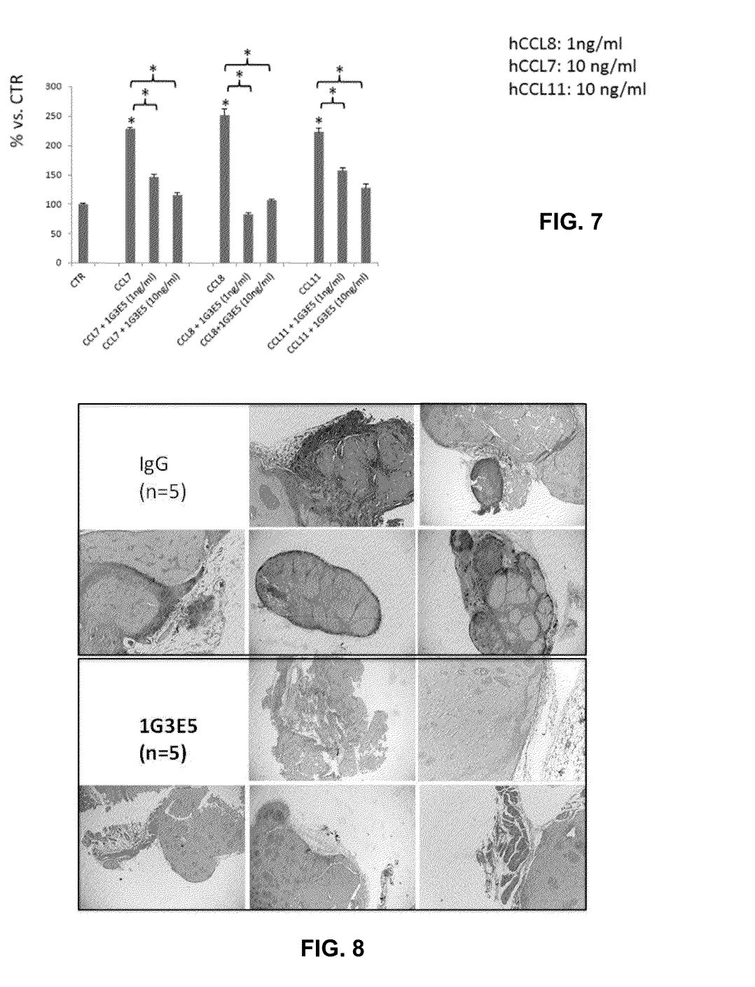

The 1 G3E5 clone was further examined for neutralizing effect on MDA-MB 231 breast cancer cell migration induced by human CCL7, CCL8, and CCL11. As shown in FIG. 7, this clone inhibited migration induced by all three cytokines.

Morphological examination of tissue (FIG.8) illustrated that the 1 G3E5 antibody completely inhibited the migration and invasion of MDA-MB 231 bGal cancer cells toward adjacent matrigel nodules containing CCL8-producing HFF2 fibroblasts. As can be seen, the MDA-MB 231 cells (darker in the images) are completely absent from the 1 G3E5-treated group (lower panels).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A method for preventing the migration of breast cancer cells, comprising inhibiting the activity of CCL8 in an area comprising the breast cancer cells.

2. The method of claim 1, the method including delivering an anti-CCL8 antibody to the area.

3. The method of claim 1, the area comprising CCL8-expressing stromal cells.

4. The method of claim 3, wherein the CCL8-expressing stromal cells comprise fibroblasts.

5. The method of claim 3, the method including inhibiting expression of the CCL8.

6. The method of claim 1, the breast cancer cells comprising estrogen-independent breast cancer cells.

7. The method of claim 6, the breast cancer cells comprising triple-negative breast cancer cells.

8. The method of claim 1, further comprising: determining the level of CCL8 in the area prior to inhibiting the activity of the CCL8 in the area.

9. A method for treatment of breast cancer in a subject, comprising inhibiting the activity of CCL8 in the subject.

10. The method of claim 9, the method including administering to the subject a therapeutically effective amount of an anti-CCL8 antibody.

11. The method of claim 9, wherein said therapeutically effective amount is between about 1 ng/kg body weight/day and about 100 mg/kg body weight/day.

12. The method of claim 9, further comprising administering a chemotherapeutic agent to the subject.

13. The method of claim 9, further comprising determining the level of CCL8 in the subject prior to inhibiting the activity of the CCL8 in the subject, the determination providing information with regard to epithelial mesenchymal transition of the breast cancer.

14. The method of claim 9, the method comprising inhibiting expression of CCL8 in the subject.

15. The method of claim 9, wherein the subject is diagnosed with an estrogen-independent breast cancer.

16. The method of claim 15, wherein the subject is diagnosed with triple-negative breast cancer.

17. A pharmaceutical composition, comprising an agent for inhibiting the activity of CCL8 and a pharmaceutically acceptable carrier.

18. The pharmaceutical composition of claim 17, wherein the agent is an anti-CCL8 antibody.

19. The pharmaceutical composition of claim 18, wherein the anti-CCL8 antibody is a monoclonal antibody.

20. The pharmaceutical composition of claim 18, wherein the anti-CCL8 antibody is specific for an antigen comprising SEQ ID NO: 1.
